(19) United States
(12) Patent Application Publication
(10) Pub. No.: US 2026/0325570 A1
(43) Pub. Date: Oct. 22, 2026

(54) METHOD FOR ENHANCING THE EFFECT OF IMMUNE CHECKPOINT INHIBITORS ON SOLID CANCER

(71) Applicants: TAIYO KAGAKU Co., Ltd., Yokkaichi-shi (JP); SHOWA Medical University, Tokyo (JP)

(72) Inventors: Takuya TSUNODA, Tokyo (JP); Atsushi HORIIKE, Tokyo (JP); Aya ABE, Yokkaichi-shi (JP); Norio SATO, Yokkaichi-shi (JP)

(21) Appl. No.: 19/453,129

(22) Filed: Jan. 20, 2026

(30) Foreign Application Priority Data
Jan. 21, 2025 (JP) 2025-008173

(51) Int. Cl. A61K 31/736; A61K 9/00; A61K 39/00; A61K 39/395; A61P 35/00

(52) U.S. Cl. CPC A61K 31/736; A61K 9/0019; A61K 9/0053; A61K 39/3955; A61P 35/00; A61K 2039/505; A61K 2039/54

(57) ABSTRACT

In a method for enhancing the efficacy of an immune checkpoint inhibitor on a solid cancer, a composition containing a guar gum hydrolysate is orally administered during administration of the immune checkpoint inhibitor. The guar gum hydrolyzate has an average molecular weight of 1.0×10³ to 1.0×10⁵, with at least 70% by weight of the guar gum hydrolyzate falling within this range. The composition has a viscosity of 10 mPa·s or less when a 2% by mass aqueous solution is measured at 37 °C using an E-type viscometer at 10 rpms. The guar gum hydrolysate is obtainable by hydrolyzing and depolymerizing, using a microbial β-mannanase, a galactomannan polysaccharide obtained from guar endosperm and having a galactose to mannose ratio (galactose: mannose) in the range of 1:1.3 to 1:2. The composition contains at least 70% by mass of dietary fiber as determined by enzymatic HPLC.

### TECHNICAL FIELD

The present invention relates to a method for enhancing the effect of immune checkpoint inhibitors on a solid cancer.

### BACKGROUND ART

Cancer causes approximately 10 million deaths worldwide annually. In recent years, several immune checkpoint inhibitors have been developed, successfully increasing the lifespan of some cancer patients and, in some cases, achieving permanent remission. However, some cancer patients may exhibit or develop resistance to immune checkpoint inhibitors. Therefore, compositions that enhance the effect of immune checkpoint inhibitors are desired.

Several studies on such compositions have been published (Patent Documents 1 and 2, Non-Patent Document 1). In Patent Document 1, a method that combines an immune checkpoint inhibitor with a monomicrobial oral preparation using Alistipes is disclosed. In Patent Document 2, a method that combines an immune checkpoint inhibitor with a glycopeptide derived from gastrointestinal mucin is disclosed.

In Non-Patent Document 1, it is disclosed that, among melanoma patients, patients who had ingested 20 g or more of dietary fiber per day at the time they were administered immune checkpoint inhibitors had the longest progression-free survival. On the other hand, probiotic supplements have been known to somewhat reduce the effectiveness of immune checkpoint inhibitors.

### PRIOR ART DOCUMENTS

### Patent Documents

Patent Document 1: JP 2023-539910 A

Patent Document 2: JP 2024-533502 A

### Non-Patent Document

Non-Patent Document 1: Dietary fiber and probiotics influence the gut microbiome and melanoma immunotherapy response, Science (2021) 374 (6575): 1632-1640

### SUMMARY OF THE INVENTION

As described above, compositions for enhancing the effects of immune checkpoint inhibitors are not yet effective in all cancer patients (especially solid cancers), and new cancer therapies are desired.

It is therefore one non-limiting object of the present teachings to provide a method that enhances the effects of immune checkpoint inhibitors on solid cancers.

The present inventors discovered that compositions containing specific guar gum hydrolyzates have the effect of enhancing the effects of immune checkpoint inhibitors on solid cancers.

Thus, a method for enhancing the effect of an immune checkpoint inhibitor on a solid cancer according to the present teachings is characterized by, during administration of the immune checkpoint inhibitor, orally administering a composition containing a guar gum hydrolyzate, the guar gum hydrolyzate having an average molecular weight of 1.0×10³ to 1.0×10⁵, with 70% or more by mass of the guar gum hydrolyzate falling within this average molecular weight range, the composition having a viscosity of 10 mPa·s or less when a 2% by mass aqueous solution is measured at 37 °C using an E-type viscometer at 10 rpm, 5 mPa·s or less when measured at 20 rpm, or 3 mPa·s or less when measured at 30 rpm, in which the composition contains guar-derived endosperm and was obtained by hydrolyzing and depolymerizing a galactomannan polysaccharide having a galactose to mannose content ratio (galactose:mannose) in the range of 1:1.3 to 1:2 using a microbial β-mannanase to reduce the molecular weight of the polysaccharide, and the composition containing at least 70% by mass of dietary fiber as determined by enzymatic HPLC.

With regard to the above composition, the daily dose is preferably 10 g to 20 g.

The above composition can be ingested by being incorporated into various foods and beverages. Therefore, foods and beverages containing the above composition can be provided.

Thus, according to the present teachings, a method for enhancing the effects of immune checkpoint inhibitors on solid cancers can be provided. In particular, a method that exhibits efficacy even for patients who have developed resistance to immune checkpoint inhibitors can be provided.

### BRIEF DESCRIPTION OF THE DRAWINGS

FIGS. 1(A)-1(D) show the results of Case 1, wherein FIG. 1(A) shows the administration schedule of an immune checkpoint inhibitor and a dietary fiber, FIG. 1(B) shows changes of tumor diameter, FIG. 1(C) shows changes of blood CEA concentrations, and FIG. 1(D) shows changes of CT images.

FIGS. 2(A)-2(C) show the results of Case 2, wherein FIG. 2(A) shows the administration schedule of the immune checkpoint inhibitor and the dietary fiber, FIG. 2(B) shows changes of tumor diameter, and FIG. 2(C) shows changes of blood CEA concentrations.

FIGS. 3(A)-3(C) show the results of Case 3, wherein FIG. 3(A) shows the administration schedule of the immune checkpoint inhibitor and the dietary fiber, FIG. 3(B) shows changes of tumor diameter, and FIG. 3(C) shows changes of blood CEA concentrations.

FIGS. 4(A)-4(C) show the results of Case 4, wherein FIG. 4(A) shows the administration schedule of the immune checkpoint inhibitor and the dietary fiber, FIG. 4(B) shows changes of tumor diameter, and FIG. 4(C) shows changes of blood CEA concentrations.

FIGS. 5(A)-5(C) shows the results of Case 5, wherein FIG. 5(A) shows the administration schedule of the immune checkpoint inhibitor and the dietary fiber, FIG. 5(B) shows changes of tumor diameter, and FIG. 5(C) shows changes of blood CEA concentrations.

FIGS. 6(A)-6(F) show the results of Case 6, wherein FIG. 6(A) shows the administration schedule of the immune checkpoint inhibitor and the dietary fiber, FIG. 6(B) shows changes of tumor diameter, FIG. 6(C) shows changes of blood CEA concentrations, FIG. 6(D) shows changes of blood CA19-9 concentration, FIG. 6(E) shows changes of Virchow lymph node images according to a CT scan, and FIG. 6(F) shows changes of para-aortic lymph node images according to CT scans.

### DETAILED DESCRIPTION OF THE INVENTION

Next, embodiments of the present teachings will be described with reference to figures. The technical scope of the present invention is not limited to these embodiments, and various embodiments can be implemented without changing the gist of the invention.

“Guar gum” refers to a water soluble natural polysaccharide obtained from the endosperm (or more precisely, cotyledons) of guar beans, which is a polysaccharide consisting of two linearly linked mannose molecules and one galactose side chain; the average molecular weight of naturally obtained guar gum is approximately 2.0×10⁵ to 3.0×10⁵. Guar gum is known to have physiological effects such as suppressing blood glucose levels, lowering cholesterol, and improving bowel movements. In the present teachings, “guar gum hydrolyzate” refers to a water soluble dietary fiber obtained by hydrolyzing and depolymerizing the galactomannan polysaccharide contained in the endosperm of guar (scientific name: Cyanopsis tetragonoloba), an annual legume plant served as food in India, Pakistan, and other countries. As methods for hydrolyzing guar gum, there are enzymatic hydrolysis, acidic hydrolysis, etc. Although there are no particular limitations, enzymatic hydrolysis is preferred because it facilitates the regulation of the molecular weight of the hydrolyzed product.

As the enzyme used in the enzymatic degradation method, although there are no particular limitations as long as it hydrolyzes linear mannose chains, it is preferred to use a β-mannanase derived from fungi such as Aspergillus or Rhizops. The upper limit of the average molecular weight distribution of the guar gum hydrolyzed product is 1.0×10⁵ or less, preferably 5.0×10⁴ or less, and more preferably 2.5×10⁴ or less. The lower limit of the average molecular weight distribution of the guar gum hydrolyzed product is 1.0×10³ or more, preferably 2.0×10³ or more. If the average molecular weight distribution is less than 1.0×10³, it becomes difficult to fully exhibit the physiological effects; if the average molecular weight exceeds 1.0×10⁵, the viscosity becomes too high, making it difficult to incorporate into foods and beverages. Although there are no particular limitations on the method for measuring the molecular weight distribution, an example is gel filtration chromatography using polyethylene glycols (average molecular weights: 2×10², 2×10³, 2×10⁴, and 1×10⁵) as the molecular weight markers.

The guar gum hydrolyzate of the present teachings is one in which 70% by mass or more, and preferably 80% by mass or more, of the guar gum hydrolyzate falls within the above average molecular weight range.

Galactose is a type of monosaccharide classified as an aldohexose; its molecular formula is C₆H₁₂O₆ and the molecular weight is 180 (both are the same as glucose). The configuration is the same at the 2nd position (second from the top in the Fischer projection), with the —OH at the 5th position pointing in the same direction, and the —OH at the 3rd and 4th positions pointing in the opposite direction, so that the configuration at the 5th position of D-galactose is the same as that of D-glyceraldehyde.

Mannose is a type of monosaccharide classified as an aldohexose; its molecular formula is C₆H₁₂O₆ and the molecular weight is 180 (both are the same as glucose). The configuration is the same at the 2nd and 3rd positions, with the —OH at the 4th and 5th positions pointing in the opposite direction, and the configuration at the 5th position of D-mannose is the same as that of D-glyceraldehyde. Mannose is not metabolized significantly by humans, and when ingested orally, very little of it enters the glycolytic pathway.

Anti-PD-1 antibodies such as nivolumab and pembrolizumab, anti-PD-L1 antibodies such as atezolizumab, durvalumab, and avelumab, and anti-CTLA-4 antibodies such as ipilimumab and tremelimumab, can be exemplified as immune checkpoint inhibitors of the present teachings.

Solid cancers of the present teachings refer to at least one selected from the group consisting of lung cancer, urothelial cancer, colorectal cancer, prostate cancer, ovarian cancer, pancreatic cancer, breast cancer, bladder cancer, gastric cancer, gastroesophageal junction adenocarcinoma, gastrointestinal stromal tumor, cervical cancer, esophageal cancer, squamous cell carcinoma, peritoneal cancer, liver cancer, hepatocellular carcinoma, endometrial cancer, uterine cancer, salivary gland cancer, kidney cancer, vulvar cancer, thyroid cancer, penile cancer, neuroepithelial tumor, head and neck cancer, skin cancer, pharyngeal cancer, gallbladder cancer, bile duct cancer, mesothelioma, Behcet's disease, and sarcoma.

When administering the immune checkpoint inhibitor according to the present teachings to humans, it may be administered, for example, at a dose of 0.001 to 100 mg/kg once or once every four weeks, or once every week, every two weeks, every three weeks, every five weeks, every six weeks, every seven weeks, or every eight weeks. It is also preferable to administer by intravenous drip at the intervals and dosages mentioned above.

When administering the guar gum hydrolyzate of the present teachings to humans, it can be administered orally at a dose of, for example, 0.05 g/kg/day to 1 g/kg/day, in accordance with the administration interval of the immune checkpoint inhibitor, and the number of administrations per day can be divided into multiple doses, such as three times.

In the present teachings, viscosity was measured using an E-type viscometer manufactured by Toki Sangyo Co., Ltd. (using a standard cone rotor 1°34′×R24). The galactose to mannose content ratio (galactose: mannose) was calculated from the measured values obtained by subjecting the acid hydrolyzed guar gum decomposition product of the present teachings to high-performance liquid chromatography. The operating conditions for the high-performance liquid chromatography are as follows.
Detector: Fluorescence detector
Column: Tosoh Corporation TSKgel® Sugar AXI, φ4.6 mm×150 mm
Column temperature: 60 °C
Mobile phase: 0.5 mol/L borate buffer (pH 8.7)
Flow rate: 0.4 mL/min
Fluorescence excitation wavelength: 320 nm
Fluorescence measurement wavelength: 430 nm
Post-column: Reaction solution: 1% L-arginine solution
   Reaction solution flow rate: 0.7 mL/min
   Reaction temperature: 150 °C

### PREPARATION OF GUAR GUM HYDROLYZATE

### Example 1

After 900 g of water was adjusted to pH 3.4 with 0.1 N hydrochloric acid, 0.24 g of a commercially available β-mannanase derived from Aspergillus sp. and 100 g of guar gum powder were added and mixed. This mixture was reacted at 45 °C to 50 °C for 20 hours. After the reaction, the enzyme was inactivated by heating at 90 °C for 15 minutes. The reaction mixture was filtered by suction to remove the insoluble matter, and the resulting clear solution was concentrated under reduced pressure (Yamato evaporator). The solid content was 21% by mass. It was dried using a spray dryer (Okawara Kakoki Co., Ltd.) to obtain 66 g of guar gum hydrolyzate as a powder.

The guar gum hydrolyzate was dissolved in water to obtain a 0.5% (w/v) aqueous solution. The average molecular weight was found to be approximately 2.3×10³ using gel filtration chromatography (column: YMC-Pack Diol-120, detector: differential refractometer) employing polyethylene glycols (average molecular weights: 2×10², 2×10³, 2×10⁴, and 1×10⁵) as molecular weight markers. It contained more than 70% by mass of polymers having molecular weights of 1.0×10³ to 1.0×10⁵.

In addition, the viscosity of a 2% by mass aqueous solution was measured at 37 °C and 20 rpm using an E-type viscometer, and was found to be 1.0 mPa·s.

The galactose to mannose content ratio (galactose:mannose) was measured, and was found to be 1:1.3.

The dietary fiber content was measured using the enzymatic HPLC method, and was found to be 72% by mass.

### Example 2

After 900 g of water was adjusted to pH 4.8 with 0.1 N hydrochloric acid, 0.12 g of a commercially available β-mannanase derived from Aspergillus sp. and 100 g of guar gum powder were added and mixed. This mixture was reacted at 45 °C to 50 °C for 10 hours. After the reaction, the enzyme was inactivated by heating at 90 °C for 15 minutes. The reaction mixture was separated by suction filtration to remove the insoluble matter, and the resulting clear solution was concentrated under reduced pressure (Yamato evaporator). The solid content was 19% by mass. It was dried using a spray dryer (Okawara Kakoki Co., Ltd.) to obtain 66 g of guar gum hydrolyzate as a powder.

The average molecular weight of the guar gum hydrolyzate was determined as in Example 1 and was found to be approximately 8.1×10⁴. It contained more than 80% by mass of polymers having molecular weights of 1.0×10³ to 1.0×10⁵ according to the result of the HPLC chart.

In addition, the viscosity of a 2% by mass aqueous solution was measured at 37 °C and 10 rpm using an E-type viscometer, and was found to be 8.1 mPa·s.

The galactose to mannose content ratio (galactose:mannose) was measured, and was found to be 1:1.8.

The dietary fiber content was measured using the enzyme-HPLC method, and was found to be 92% by mass.

### Example 3

After 900 g of water was adjusted to pH 4.2 with 0.1 N hydrochloric acid, 0.3 g of a commercially available β-mannanase derived from Aspergillus sp. and 100 g of guar gum powder were added and mixed. This mixture was reacted at 50 °C to 55 °C for 24 hours. After the reaction, the enzyme was inactivated by heating at 90 °C for 15 minutes. The reaction mixture was filtered under suction to remove insoluble matter, and the resulting clear solution was concentrated under reduced pressure (Yamato evaporator). The solid content was 21% by mass. It was then dried using a spray dryer (Okawara Kakoki Co., Ltd.) to obtain 67 g of guar gum hydrolyzate as a powder.

The average molecular weight of the guar gum hydrolyzate was determined as in Example 1 and was found to be approximately 1.3×10⁴. It contained more than 80% by mass of polymers having molecular weights between 1.0×10³ and 1.0×10⁵ according to the result of the HPLC chart.

In addition, the viscosity of a 2% by mass aqueous solution was measured at 37 °C and 30 rpm using an E-type viscometer, and was found to be 1.6 mPa·s.

The galactose to mannose ratio (galactose:mannose) was measured, and was found to be 1:2.0.

The dietary fiber content was measured using an enzymatic HPLC method and was found to be 90% by mass.

### Example 4

After 900 g of water was adjusted to pH 3.8 with 0.1 N hydrochloric acid, 0.36 g of a commercially available β-mannanase derived from Aspergillus sp. and 100 g of guar gum powder were added and mixed. This mixture was reacted at 50 °C to 55 °C for 20 hours. After the reaction, the enzyme was inactivated by heating at 90 °C for 15 minutes. The reaction mixture was separated by suction filtration to remove the insoluble matter, and the resulting clear solution was concentrated under reduced pressure (Yamato evaporator). The solid content was 21% by mass. It was dried using a spray dryer (Okawara Kakoki Co., Ltd.) to obtain 68 g of guar gum hydrolyzate as a powder.

The average molecular weight of the guar gum hydrolyzate was determined as in Example 1, and was found to be approximately 4.8×10³. It contained more than 80% by mass of polymers having molecular weights of 1.0×10³ to 1.0×10⁵ according to the result of the HPLC chart.

In addition, the viscosity of a 2% by mass aqueous solution was measured at 37 °C and 20 rpm using an E-type viscometer, and was found to be 1.2 mPa·s.

The galactose to mannose content ratio (galactose:mannose) was measured, and was found to be 1:1.7.

The dietary fiber content was measured using the enzymatic HPLC method, and was found to be 90% by mass.

### Test Method

The subjects were patients with advanced solid cancers (non-small cell lung cancer (5 cases), gastric cancer (5 cases), and esophageal cancer (2 cases)) that had acquired resistance to cancer immunotherapy. 480 mg of an immune checkpoint inhibitor (nivolumab) was administered intravenously to each subject every four weeks. In conjunction with the nivolumab administration period, the composition described in Example 1 (guar gum hydrolyzate) was orally administered 15 g/day (5 g×3 times).

During the study period, chest and abdominal CT scans were taken at 8, 16, and 26 weeks (and as appropriate thereafter) to evaluate efficacy according to RECIST criteria; evaluation of safety was performed by medical examinations and blood tests every four weeks.

In addition, if any serious adverse event of grade 3 or higher was observed in the CTC-AE during the study period and the attending physician also decided it would be medically inappropriate to continue the study for that patient, or if progression of disease was observed according to the RECIST criteria, a judgement was made “cancer has progressed (progressive disease: PD)”; furthermore, if the attending physician determined that there would be no clinical benefit to the patient, the study was discontinued for that patient. International standards exist for assessing the effectiveness of cancer treatment. Specifically, CR (complete response) indicates a complete response, PR (partial response) indicates a partial response, SD (stable disease) indicates a stable state (limited shrinkage or growth) that is neither CR nor PD, and PD (progressive disease) indicates progression with a 20% or greater increase in the disease or the appearance of new lesions.

The following analysis method was used. The expected 6-month progression-free survival rate was set at 35%, the threshold 6-month progression free survival rate was set at 5%, and the statistical power was set at 70% for a one-sided test with a significance level of α=0.1; using the two-step method (Minimax design) of Simon, the required number of cases was calculated to be seven. Each cohort will be evaluated in two stages; the first stage will be performed when four cases have been collected; if none of the four cases survive for six months without progression, the therapy will be deemed ineffective in that cohort and enrollment will be discontinued; if one or more of the four cases survive for six months without progression, enrollment will continue. In the second stage, if two or more of the seven cases survive for six months without progression, the therapy will be deemed effective in that cohort.

### Study Results

The results of all 12 cases are summarized as follows. For lung cancer patients, one patient achieved PR and four patients achieved SD. Three of the five patients achieved 6-month progression-free survival and were deemed effective. The response rate was 20%, and the disease control rate was 100%.

For gastric cancer patients, two patients achieved PR and two patients achieved SD. Two of the five patients achieved progression-free survival and were deemed effective. The response rate was 50%, and the disease control rate was 100%.

For esophageal cancer, the timeframe for evaluation had not yet been reached.

Six representative cases are presented below.

### Case 1

Case 1 was a 73-year-old male patient with lung cancer (adenocarcinoma) (stage IVB, PD-L1 (TPS 10-20%)). With regard to the patient's treatment history, chest radiation therapy from Mar. 17, 2017 to Apr. 13, 2017, combination therapy of carboplatin (CBDCA) with pemetrexed (PEM) from Nov. 22, 2017 to May 12, 2018, treatment with pembrolizumab (an immune checkpoint inhibitor) from Jun. 19, 2018 to Oct. 26, 2020, and treatment with atezolizumab (an immune checkpoint inhibitor) from Jul. 11, 2023 to Feb. 20, 2024, had been received; acquired resistance to both of these immune checkpoint inhibitors was observed.

The treatment described in the above study method was received starting on Mar. 19, 2024.

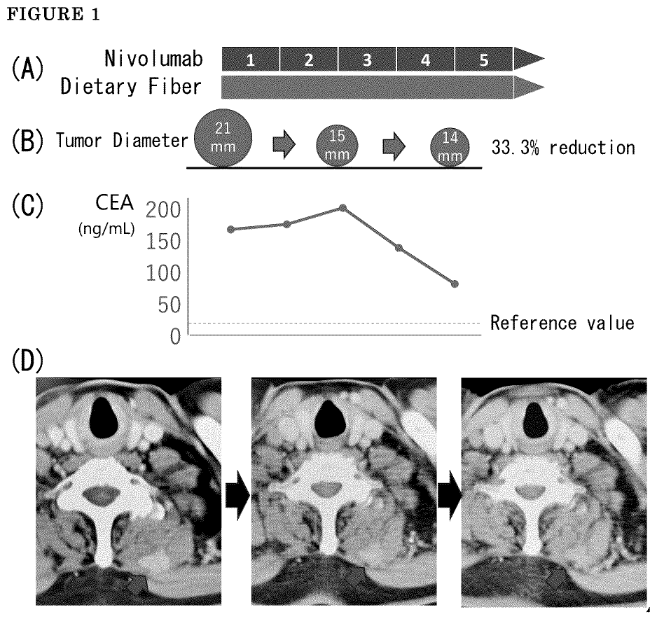

The results are shown in FIG. 1. The tumor diameter was confirmed to have decreased 33.3% from 21 mm at the start of the study, to 15 mm eight weeks later and 14 mm 16 weeks later, and the anti-tumor effect was PR. Furthermore, CEA levels decreased from 165 ng/ml to 75 ng/ml based on measurements every four weeks from the start of the study. Efficacy was maintained even in the CT evaluation at 26 weeks, and the primary endpoint of this study, six month progression-free survival, was achieved.

Thus, in the present case, in a patient who had acquired resistance to immune checkpoint inhibitors, it was found that the efficacy of an immune checkpoint inhibitor was significantly enhanced by the combined use of the guar gum hydrolyzate with the immune checkpoint inhibitor.

### Case 2

Case 2 was a 78-year-old female patient with recurrent lung cancer (adenocarcinoma) after surgery (EGFR+, PD-L1 (TPS<1%)). With regard to the patient's treatment history, treatment with afatinib (an EGFR tyrosine kinase inhibitor) from Apr. 20, 2017 to Jun. 23, 2019, and treatment with atezolizumab (an immune checkpoint inhibitor), bevacizumab (an anti-VEGF antibody), carboplatin, and paclitaxel (ABCP) from Jul. 18, 2019 to Feb. 27, 2023, had been received; acquired resistance to atezolizumab was observed.

The treatment described in the above study method was received starting on Feb. 5, 2024.

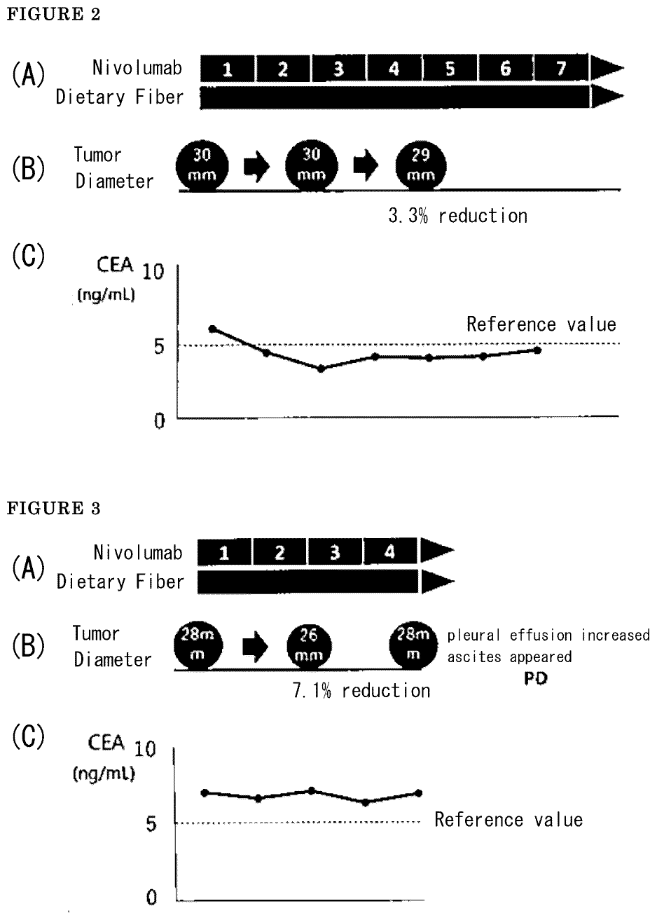

The results are shown in FIG. 2. The tumor diameter was confirmed to have decreased by 3.3% from 30 mm at the start of the study, to 30 mm 8 weeks later and to 29 mm 16 weeks later; the antitumor effect was found to be SD. Furthermore, CEA levels decreased slightly from 6.5 ng/ml before the start of the study to 4.8 ng/ml six weeks after the start of the study based on measurements every four weeks from the start of the study. Efficacy was maintained even in the CT evaluation at 26 weeks, and the primary endpoint of this study, six month progression-free survival, was achieved.

Thus, in the present case, in a patient who had acquired resistance to an immune checkpoint inhibitor, it was found that the progression of recurrent lung cancer was suppressed by the combined use of the immune checkpoint inhibitor with the guar gum hydrolyzate.

### Case 3

Case 3 was a 77-year-old female patient with recurrent lung cancer (adenocarcinoma) after surgery (EGFR mutation-positive, PD-L1 (TPS 1-49%)). With regard to the patient's treatment history, treatment with afatinib (EGFR tyrosine kinase inhibitor) from Mar. 9, 2017 to Sep. 29, 2022, and ABCP therapy from Oct. 20, 2022 to Jul. 24, 2023, had been received; acquired resistance to atezolizumab was observed.

The treatment described in the above study method was received starting on Mar. 4, 2024.

The results are shown in FIG. 3. The tumor diameter was confirmed to have decreased 7.1% from 28 mm at the start of the study, to 26 mm 8 weeks later and 26 mm 16 weeks later. CEA levels changed from 7.0 ng/ml before the start of the study to 6.8 ng/mL 16 weeks later based on measurements every four weeks from the start of the study.

In the present case, after 16 weeks from the start of the study, the patient's pleural effusion increased and ascites appeared, so the cancer was considered to have progressed and the study was terminated.

### Case 4

Case 4 was a 60-year-old male patient with recurrent lung cancer (adenocarcinoma) after surgery (EGFR gene mutation positive, PD-L1 (TPS 1-10%)). With regard to the patient's treatment history, treatment with gefitinib (EGFR tyrosine kinase inhibitor) from Feb. 5, 2013 to May 17, 2021, and ABCP therapy from Jun. 2, 2021 to Jun. 12, 2023, had been received; acquired resistance to atezolizumab was observed.

The treatment described in the above study method was received starting on Mar. 1, 2024.

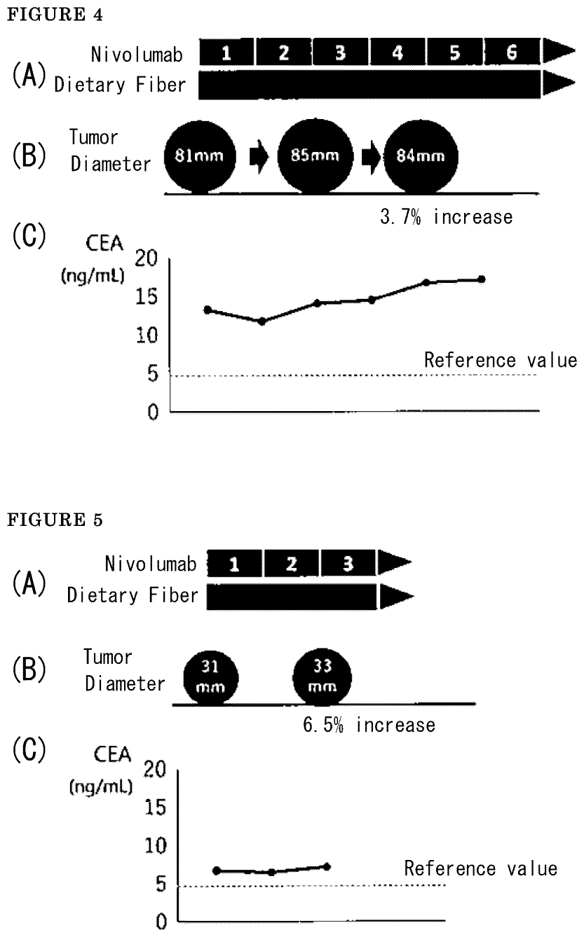

The results are shown in FIG. 4. The tumor diameter was confirmed to have increased slightly by 3.7% from 81 mm at the start of the study, to 85 mm 8 weeks later and 84 mm 16 weeks later. CEA levels changed from 14.0 ng/ml before the start of the study to 16.5 ng/ml 5 weeks later based on measurements every four weeks from the start of the study. Efficacy was maintained even in the CT evaluation at 26 weeks, and the primary endpoint of this study, six month progression-free survival, was achieved. In the present case, in a patient who had acquired resistance to an immune checkpoint inhibitor, significant progression of recurrent lung cancer was suppressed by the combined use of the immune checkpoint inhibitor with the guar gum hydrolyzate.

### Case 5

Case 5 was a 76-year-old female patient with recurrent gastric cancer after surgery. With regard to the patient's treatment history, treatment with capecitabine (an anticancer drug) from Jul. 27, 2018 to Apr. 27, 2020, treatment with albumin-bound paclitaxel (nab-PTX) and ramucirumab (RAM) from May 11, 2020 to Aug. 25, 2020, and treatment with nivolumab (an immune checkpoint inhibitor) from Oct. 13, 2020 to Apr. 30, 2024, had been received; acquired resistance to nivolumab was observed.

The treatment described in the above study method was received starting on Jun. 11, 2024.

The results are shown in FIG. 5. The tumor diameter was confirmed to have increased by 6.5% from 31 mm at the start of the study to 33 mm eight weeks later; the antitumor effect was found to be SD. CEA levels changed from 7.0 ng/ml before the start of the study to 7.5 ng/ml eight weeks later based on measurements every four weeks from the start of the study.

In the present case, after eight weeks from the start of the study, the tumor diameter increased by 6.5%, and the antitumor effect was found to be SD Efficacy was maintained in the CT evaluation at 26 weeks, and the primary endpoint of this study, six-month progression free survival, was achieved.

### Case 6

Case 6 was a 55-year-old female patient with recurrent gastric cancer (HER2-negative) after surgery. With regard to the patient's treatment history. SOX therapy from Nov. 20, 2019, to Feb. 5, 2020, had been received and acquired resistance was observed.

From Feb. 27, 2020, to Feb. 20, 2023, treatment with nivolumab had been received, and acquired resistance was observed.

The treatment described in the above study method was received starting on Feb. 17, 2025.

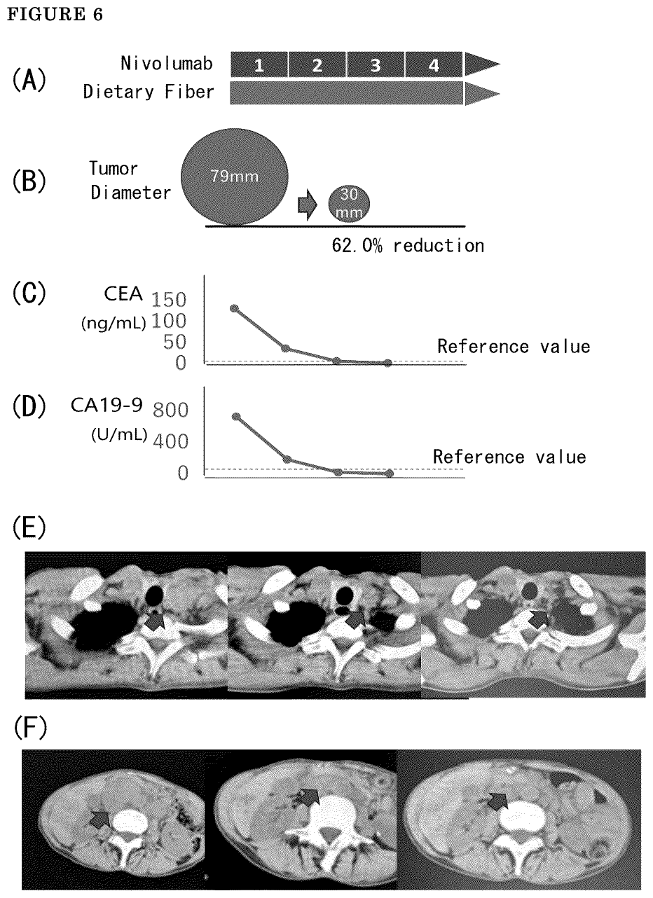

The results are shown in FIG. 6. The tumor diameter was confirmed to have decreased by 62% from 79 mm at the start of the study to 30 mm 8 weeks later. The antitumor effect was Response. CEA decreased from 130 ng/mL to below the reference value based on measurements every four weeks from the start of the study. CA 19-9 decreased from 700 U/mL to below the reference value based on measurements every four weeks from the start of the study. Efficacy was maintained even in the CT evaluation at 26 weeks, and the primary endpoint of this study, six month progression-free survival, was achieved.

In the present case, in a patient who had acquired resistance to an immune checkpoint inhibitor, it was found that the effect of the immune checkpoint inhibitor was significantly enhanced by the combined use of the immune checkpoint inhibitor with the guar gum hydrolyzate.

As shown in the examples above, in patients who had acquired resistance to immune checkpoint inhibitors, cases were observed, which showed a significant inhibitory effect on tumor progression for recurrent solid cancers owing to the combined use of an immune checkpoint inhibitor with a guar gum hydrolyzate.

In this way, according to the present embodiments, a method for enhancing the effect of immune checkpoint inhibitors on solid cancers can be provided. In particular, even in patients who have acquired resistance to immune checkpoint inhibitors, a method that showed effectiveness can be provided. The composition of the present embodiment can be administered by being incorporated into various foods and beverages.

1. A method for enhancing the efficacy of an immune checkpoint inhibitor on a solid cancer, comprising:
during administration of the immune checkpoint inhibitor, orally administering a composition containing a guar gum hydrolyzate,
wherein:
the guar gum hydrolyzate has an average molecular weight of 1.0×10³ to 1.0×10⁵, with at least 70% by weight of the guar gum hydrolyzate falling within this average molecular weight range,
the composition has a viscosity of 10 mPa·s or less when a 2% by mass aqueous solution is measured at 37 °C using an E-type viscometer at 10 rpms, or 5 mPa·s or less when measured at 20 rpm, or 3 mPa·s or less when measured at 30 rpm, the guar gum hydrolysate was obtained by hydrolyzing and depolymerizing, using a microbial β-mannanase, a galactomannan polysaccharide obtained from guar endosperm and having a galactose to mannose ratio (galactose:mannose) in the range of 1:1.3 to 1:2, and
the composition contains at least 70% by mass of dietary fiber as determined by enzymatic HPLC.

2. The method according to claim 1, wherein the guar gum hydrolysate is orally administered daily with a dosage of 10 g to 20 g.

3. The method according to claim 1, wherein the microbial β-mannanase is derived from a fungus.

4. The method according to claim 1, wherein the microbial β-mannanase is derived from Aspergillus.

5. The method according to claim 1, wherein the average molecular weight of the guar gum hydrolysate is 1.0×10³ to 2.5×10⁴.

6. The method according to claim 1, wherein the immune checkpoint inhibitor is selected from the group consisting of an anti-PD-1 antibody, an anti-PD-L1 antibody and an anti-CTLA-4 antibody.

7. The method according to claim 1, wherein the guar gum hydrolysate is orally administered at a dosage of 0.05 g/kg/day to 1 g/kg/day.

8. The method according to claim 1, wherein the immune checkpoint inhibitor is administered intravenously.

9. The method according to claim 1, wherein the immune checkpoint inhibitor is nivolumab.

10. The method according to claim 1, wherein the solid cancer is lung cancer.

11. The method according to claim 1, wherein the solid cancer is gastric cancer.

12. The method according to claim 7, wherein the immune checkpoint inhibitor is selected from the group consisting of an anti-PD-1 antibody, an anti-PD-L1 antibody and an anti-CTLA-4 antibody.

13. The method according to claim 12, wherein the average molecular weight of the guar gum hydrolysate is 1.0×10³ to 2.5×10⁴.

14. The method according to claim 13, wherein the immune checkpoint inhibitor is selected from the group consisting of an anti-PD-1 antibody, an anti-PD-L1 antibody and an anti-CTLA-4 antibody.

15. The method according to claim 14, wherein the immune checkpoint inhibitor is administered intravenously.

16. The method according to claim 15, wherein the immune checkpoint inhibitor is nivolumab.

17. The method according to claim 16, wherein the solid cancer is lung cancer.

18. The method according to claim 16, wherein the solid cancer is gastric cancer.

19. The method according to claim 18, wherein the microbial β-mannanase is derived from Aspergillus.

20. The method according to claim 17, wherein the microbial β-mannanase is derived from Aspergillus.
